(19) United States
(12) Patent Application Publication
(10) Pub. No.: US 2023/0375554 A1
(43) Pub. Date: Nov. 23, 2023

(54) IDENTIFICATION AND CHARACTERIZATION OF OVARIAN CANCER SPECIFIC BIOMARKERS IN VAGINAL SECRETIONS

(71) Applicant: GINA-LIFE DIAGNOSTICS LTD., Haifa (IL)

(72) Inventors: Inbal ZAFIR-LAVIE, Kiryat Atta (IL); Shlomit YEHUDAI-RESHEF, Habonim (IL); Maria DOLINSKI, Karmiel (IL)

(21) Appl. No.: 18/030,353

(22) PCT Filed: Oct. 4, 2021
(86) PCT No.: PCT/IL2021/051190

§ 371 Date: Apr. 5, 2023

Related U.S. Application Data
(60) Provisional application No. 63/162,633, filed on Mar. 18, 2021.
(60) Provisional application No. 63/108,886, filed on Nov. 3, 2020.
(60) Provisional application No. 63/087,307, filed on Oct. 5, 2020.

(51) Int. Cl. G01N 33/574

(52) U.S. Cl. CPC G01N 33/57449

(57) ABSTRACT

Provided herein are methods and kits for detection and identification of ovarian cancer in subject, based on determination of the levels of specific biomarkers in vaginal secretions of the subject. In particular, the methods and kits disclosed herein allow early stage detection of ovarian cancer based on levels of specific biomarkers in vaginal secretion of the subjects.

### FIELD OF INVENTION

The disclosure generally relates to biomarkers in vaginal secretions, kits for their detection and uses thereof for diagnosis and screening of ovarian cancer.

### BACKGROUND

Epithelial ovarian cancer accounts for 2.5% of all cancers in the female population and is considered one of the deadliest cancers despite the progress in technology and medical research. Most ovarian tumors appear in postmenopausal women. Unfortunately, most women are diagnosed at advanced stages (such as stage 3) of the disease, rendering ovarian cancer a “silent killer”.

Ninety percent of ovarian cancers are from a source of epithelial cells that surround the ovary. Risk factors for malignant change in these cells are hormonal changes, family history of ovarian cancer, breast cancer or colon cancer and genetic predisposition. Women carrying the BRCA1/BRCA2 genetic mutation have a 60% chance of developing ovarian cancer.

Currently, the only effective way to detect ovarian cancer is through a combination of ultrasound imaging and increased CA-125 levels in serum. However, high CA-125 levels are not specific to ovarian tumors, and its level can also increase as a result of other diseases such as endometriosis and pelvic inflammatory disease. Furthermore, it has been found that about 25% of women who suffer from ovarian cancer Stage 1 has a normal serum CA125 level and in women with multiple benign conditions CA125 level are often high making CA125 an unreliable marker.

Moreover, the diagnosis of the epithelial ovarian cancer is usually made at late stages of the disease since the clinical symptoms are few and elusive at the early stages of the disease. Accordingly, whereas CA-125 level in serum is relatively effective in predicting the response of a patient to treatment and in identifying recurrence of the disease, it does not aid in detecting early-stage ovarian malignancy.

U.S. Pat. No. 9,274,118 is directed to predicting the presence, subtype and stage of ovarian cancer, as well as for assessing the therapeutic efficacy of a cancer treatment and determining whether a subject potentially is developing cancer. Associated test kits, computer and analytical systems as well as software and diagnostic models are also provided.

International patent publication no. WO/2020/152679 is directed to a female hygienic device for diagnosing and/or monitoring cancer, comprising at least one absorption zone for accumulating vaginal discharge, and at least one agent for visually reacting with a physiological marker, the physiologic marker is indicative of a cancerous condition.

Additional background art includes U.S. Pat. No. 9,918,702, U.S. patent Ser. No. 10/605,811, U.S. Pat. No. 8,682,591, US Patent Publication No. 0150031561, U.S. Pat. No. 8,206,934, US Patent Publication No. 0150045243, U.S. Pat. Nos. 7,270,960, 8,486,648, US Patent Publication No. 0190125316, US Patent Publication No. 0190119734, US Patent Publication No. 0120309645, US Patent Publication No. 0130330747.

Nevertheless, there is a need in the art for accurate, safe, cost effective, non-invasive and reliable methods and kits for detection of ovarian cancer at various stages thereof, by identifying specific biomarkers in vaginal secretions.

### SUMMARY OF THE INVENTION

According to some embodiments, provided herein are advantageous methods and kits for detection of ovarian cancer in females, by identifying one or more specific biomarkers in vaginal secretions. In some embodiments, the advantageous methods and kits disclosed herein are further capable of differentiating between disease conditions (i.e., stage of cancer) by detecting the expression of one or more specific biomarkers obtained or derived from vaginal secretions of the subject.

According to some embodiments, there is provided a method for identifying or detecting cancer related condition in a female subject, based on identification of one or more specific biomarkers (a panel of biomarkers) in vaginal secretions.

In some embodiments, the presence or relative abundancy of one or more specific biomarkers is indicative of ovarian cancer condition and/or the stage of the cancer.

In some embodiments, the methods and kits disclosed herein are advantageous as they allow a safe, sensitive, efficient, cost effective, non-invasive and accurate means for determining ovarian cancer condition in a subject, based on the identification of one or more specific biomarkers in vaginal secretions of the subject. In some embodiments, the methods and kits are advantageous as they allow not only accurately detecting the presence of a caner condition, but can also provide indication as to the stage/severity of the condition. In further embodiments, the methods and kits disclosed herein are advantageous as they are non-invasive, easy for use and do not require blood/serum samples or processing thereof. In some embodiments, the methods and kits disclosed herein are further advantageous as they can provide indication regarding a preferred or recommended therapy, based on the identification (presence/abundancy) of one or more specific biomarkers in the vaginal secretions.

According to some embodiments, as exemplified herein, it was surprisingly found that the levels of various biomarkers (such as, for example, IL-2, IL-13, Midkine, TGM2 TRAIL, EpCAM, TGF, SCF, CD40 ligand, Flt-3 ligand, Fractalkine, Gelactine 9, IL-33, IL-1a, PSA, MIP-1a, MIP-1b, MIP-3b), in vaginal secretions of ovarian cancer patients is significantly higher as compare to their levels in vaginal secretions of healthy subjects, indicating them as predictors of early-stages disease.

In further embodiments, as exemplified herein, without wishing to be bound by any theory or mechanism, it was surprisingly found that the levels (in vaginal secretions) of various immune-related biomarkers rather than cancer-related biomarkers can facilitate differentiating between low and high Grade epithelial ovarian cancers (EOC) and/or low grade EOC and/or benign conditions, and can thus provide indication as to ovarian cancer related conditions at early stages thereof.

According to some embodiments, there is provided a method for detection of ovarian cancer in a subject, the method includes detecting the levels of a biomarker selected from: Interleukin 2 (IL-2), IL-1a, Interleukin 13 (IL-13), CD-40 Ligand, Flt-3 Ligand, Fractalkine, IL-33, GALECTIN-9, Midkine, MIP-1a, MIP-1b, MIP-3b, PD-L1, RANTES, TGF-alpha, TGM2, Total PSA, TRAIL, Stem Cell factor (SCF), EpCAM, Galectin-1 and/or Galectin-9, in vaginal secretion(s) of the subject.

According to some embodiments, the method includes detecting at least one or more of: TRAIL, CD40 ligand, Flt-3 Ligand, Fractalkine, Midkine, TGF-alpha, PDL-1 and/or TGM2. In some embodiments, the method may further include detecting the levels of one or more additional biomarkers selected from: AFP, PSA, CEA, SCF, CD44, Mesothelin, TGM2, EpCAM, Galectin-1, IL-1a, IL-2, IL-8, IL-13, IL-33, IP-10, MIP1a, MIP1b, MIP3b, TNF-a and/or VEGF.

According to some embodiments of the method, a change in the level of one or more of the biomarkers as compared to a control group of subjects not afflicted with cancer, is indicative of ovarian cancer in the subject.

According to some embodiments, the method may further allow differentiating between high stage epithelial ovarian cancer (EOC) and low stage epithelial ovarian cancer, based on the relative levels of one or more biomarkers.

According to some embodiments, a change in the levels of one or more of: PD-L1, IL-2, MIP-1b, CD-40 Ligand, Flt3-Ligand, TRAIL, TGF-a, IL-13, IL-33 and/or fractalkine is indicative of the stage of EOC.

According to some embodiments, the method may further include detecting the levels of at least two biomarkers. In some embodiments, the method may include detecting the levels of at least three biomarkers.

According to some embodiments, the detection may be facilitated using reagents capable of identifying the one or more biomarkers in the vaginal secretion. According to some embodiments, the reagent may be an antibody capable of specifically binding/recognizing a respective biomarker or a fragment thereof.

According to some embodiments, the method may further include providing a treatment recommendation, based on the levels of the detected one or more biomarkers in the sample obtained from the vaginal secretion of the subject. In some embodiments, the treatment recommendation may include recommendation to administer a therapeutic agent selected from: PARP inhibitor, Olaparib, Rucaparib, Niraparib, Talazoparib, cisplatin, carboplatin, paclitaxel, docetaxel, or any combination thereof.

According to some embodiments, there is provided a kit for detection of ovarian cancer in a subject, the kit includes one or more reagents capable of detecting the level of one or more of: Interleukin 2 (IL-2), IL-1a, Interleukin 13 (IL-13), CD-40 Ligand, Flt-3 Ligand, Fractalkine, IL-33, GALECTIN-9, Midkine, MIP-1a, MIP-1b, MIP-3b, PD-L1, RANTES, TGF-alpha, TGM2, Total PSA, TRAIL, Stem Cell factor (SCF), EpCAM, Galectin-1 and/or Galectin-9, in a vaginal secretion sample obtained from the subject.

According to some embodiments, the kit includes one or more reagents capable of detecting the level of at least TRAIL, CD40 ligand, Flt-3 Ligand, Fractalkine, Midkine, TGF-alpha, PDL-1 and/or TGM2 in the vaginal secretion sample. In some embodiments, the kit may include one or more additional reagents capable of detecting the levels of one or more additional biomarkers, selected from: AFP, PSA, CEA, SCF, CD44, Mesothelin, TGM2, EpCAM, Galectin-1, IL-1a, IL-2, IL-8, IL-13, IL-33, IP-10, MIP1a, MIP1b, MIP3b, TNF-a and/or VEGF.

According to some embodiments, the kit may include reagents capable of detecting the levels of at least two biomarkers. In some embodiments, the kit may include reagents capable of detecting the levels of at least three biomarkers.

According to some embodiments of the kit, wherein a detected change in the level of one or more of the biomarkers as compared to a control group of subjects (not afflicted with ovarian cancer), is indicative of ovarian cancer in the subject.

According to some embodiments, the kit may further allow differentiating between high stage epithelial ovarian cancer (EOC) and low stage epithelia ovarian cancer, based on the relative levels of one or more biomarkers.

According to some embodiments, the kit includes reagents capable of detecting a change in the levels of PD-L1, IL-2, MIP-1b, CD-40 Ligand, Flt3-Ligand, TRAIL, TGF-a, IL-13, IL-33 that is indicative of the stage of EOC.

According to some embodiments, the reagents are antibodies.

According to some embodiments, there is provided a method for detection of ovarian cancer in a subject, the method includes detecting the levels of at least two biomarkers selected from: AFP, Total PSA, TRAIL, CEA, SCF, OPN, b-HCG, TGFa, CA9, Midkine, TGM2, EpCAM, GALECTIN-1, GALECTIN-9, PD-L1, CD40 ligand, Flt-3 ligand, Fraktaline, IL-1a, IL-2, IL-13, IL-33, IP-10, MIP-1a, MIP-1b, MIP-3b, and/or TNF-a in vaginal secretion of a subject.

According to some embodiments, the at least two biomarkers may be selected from: TRAIL, TGFa, Midkine, TGM2, CD40 ligand, Flt-3 ligand, Fraktaline and/or PD-L1.

According to some embodiments, there is provided a kit for detection of ovarian cancer in a subject, the kit includes one or more reagents capable of detecting the level of at least two of: AFP, Total PSA, TRAIL, CEA, SCF, OPN, b-HCG, TGFa, CA9, Midkine, TGM2, EpCAM, GALECTIN-1, GALECTIN-9, PD-L1, CD40 ligand, Flt-3 ligand, Fraktaline, IL-1a, IL-2, IL-13, IL-33, IP-10, MIP-1a, MIP-1b, MIP-3b, and/or TNF-a in a sample obtained from vaginal secretion of the subject.

According to some embodiments, there is provided a method for detection of ovarian cancer in a subject, the method includes detecting the levels of at least one biomarker selected from: TRAIL, TGFa, Midkine, TGM2, CD40 ligand, CD-44, Flt-3 ligand, Fraktaline and/or PD-L1 and at least one additional biomarker selected from: Total PSA, MIF, Leptin, CEA, SCF, Mesothelin, EpCAM, GALECTIN-1, IL-1a, IL-2, IL-8, IL-13, IL-33, IP-10, MIP-1a, MIP-1b, MIP-3b, TNF-a, and/or VEGF.

According to some embodiments, there is provided a kit for detection of ovarian cancer in a subject, the kit includes one or more reagents capable of detecting the levels of at least one biomarker selected from: TRAIL, TGFa, Midkine, TGM2, CD40 ligand, CD-44, Flt-3 ligand, Fraktaline and/or PD-L1 and at least one additional biomarker selected from: Total PSA, MIF, Leptin, CEA, SCF, Mesothelin, EpCAM, GALECTIN-1, IL-1a, IL-2, IL-8, IL-13, IL-33, IP-10, MIP-1a, MIP-1b, MIP-3b, TNF-a, and/or VEGF in a sample of vaginal secretion obtained from thee subject.

According to some embodiments, there is provided a method for early detection of early stage epithelial ovarian cancer (EOC) in a subject, the method includes detecting the levels of one or more immune-related biomarkers in vaginal secretions of a subject, the biomarkers include at least one of: PD-L1, IL-2, MIP-1b, CD-40 Ligand, Flt3-Ligand, TRAIL, TGF-a, IL-13, IL-33 and/or fractalkine.

According to some embodiments, the method for early detection may further include detecting the levels of CD44 and/or IL-8 in the vaginal secretion.

According to some embodiments, the method for early detection may further allow differentiating between low grade EOC, high grade and/or benign conditions.

According to some embodiments, in the method for early detection a change in the level of one or more of the biomarkers as compared to a control group of subjects not afflicted with cancer, is indicative of ovarian cancer in the subject.

According to some embodiments, there is provided a kit for early detection of early stage epithelial ovarian cancer (EOC) in a subject, the kit includes reagents capable of detecting the levels of one or more immune-related biomarkers, the biomarkers include at least one of: PD-L1, IL-2, MIP-1b, CD-40 Ligand, Flt3-Ligand, TRAIL, TGF-a, IL-13, IL-33 and/or fractalkine, in a sample obtained from vaginal secretion of the subject. In some embodiments, the kit may further include reagents for detecting the levels of CD44 and/or IL-8 in vaginal secretions of a subject.

Further embodiments, features, advantages and the full scope of applicability of the present invention will become apparent from the detailed description and drawings given hereinafter. However, it should be understood that the detailed description, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

Exemplary embodiments are illustrated in referenced figures. Dimensions of components and features shown in the figures are generally chosen for convenience and clarity of presentation and are not necessarily shown to scale. The figures are listed below.

FIGS. 1A-B—Graphs showing CA125 levels (pg/mg protein) in vaginal secretions and serum in healthy control subjects (FIG. 1A) and ovarian cancer patients (FIG. 1B);

FIG. 2—Graphs showing CA125 levels as log of difference between vaginal secretions/serum in samples obtained from healthy control subjects and ovarian cancer patients;

FIGS. 3A-C—Graphs showing CA125 serum levels in healthy women, ovarian cancer patients after neo-adjuvant treatment and ovarian cancer patients without treatment (non-neoadjuvant);

FIGS. 4A-C—Graphs showing CA125 vaginal secretions levels in healthy women, ovarian cancer patients after neo-adjuvant treatment and ovarian cancer patients without treatment (non-neoadjuvant);

FIG. 5—Graphs showing the difference in CA125 levels between vaginal secretions and serum in patients who received treatment (neoadjuvant) and patients who did not receive treatment (non-neoadjuvant) as log (vaginal secretions/serum);

FIG. 6A—Graphs showing IL-2 levels in vaginal secretions of ovarian cancer patients and healthy women;

FIG. 6B—Graphs showing IL-2 levels in vaginal secretions in healthy women, ovarian cancer patients after neo-adjuvant treatment and ovarian cancer patients without treatment (non-neoadjuvant);

FIG. 7A—Graphs showing IL-13 levels in vaginal secretions of ovarian cancer patients and healthy women;

FIG. 7B—Graphs showing IL-13 levels in vaginal secretions in healthy women, ovarian cancer patients after neo-adjuvant treatment and ovarian cancer patients without treatment (non-neoadjuvant);

FIGS. 8A-8U—Graphs showing the indicated biomarker levels in vaginal secretions obtained from healthy woman subjects and ovarian cancer patients: FIG. 8A—Midkine, FIG. 8B—mesothelin; FIG. 8C—TGM2; FIG. 8D—EpCAM; FIG. 8E—MIF; FIG. 8F-TRAIL; FIG. 8G—TGF-a; FIG. 8H—SCF; FIG. 8I—CD40 Ligand; FIG. 8J—Flt3-Ligand; FIG. 8K—Fractalkine; FIG. 8L—CD44; FIG. 8M—GELACTINE 9; FIG. 8N—IL-13; FIG. 8O—IL-2; FIG. 8P—IL-33; FIG. 8Q—IL1a; FIG. 8R—PSA; FIG. 8S—MP-1; FIG. 8T—; MIP-1b; FIG. 8U—MIP-3b;

FIGS. 9A-L—Graphs showing the indicated immune-related biomarker levels in vaginal secretions obtained from high stage epithelial ovarian cancer (EOC) patients (n=18) compared to benign gynecological conditions subjects (n=18) and low grade EOC patient. FIG. 9A—CD44; FIG. 9B—IL-8; FIG. 9C—PD-L1; FIG. 9D—IL-2; FIG. 9E—MIP-1b; FIG. 9F—IL-33; FIG. 9G—IL-13; FIG. 9H—Fractalkine; FIG. 9I—FLT3-Ligand; FIG. 9J—CD-40 Ligand; FIG. 9K—TRAIL; FIG. 9L—TGF-a; and

FIGS. 10A-E—Graphs showing the indicated cancer-related biomarker levels in vaginal secretions obtained from high stage epithelial ovarian cancer (EOC) patients (n=18) compared to benign gynecological conditions subjects (n=18) and low grade EOC patient. FIG. 10A—Total PSA; FIG. 10B—CA-9; FIG. 10C—Mesothelin; FIG. 10D—TGM-2; FIG. 10E—EpCAM.

### DETAILED DESCRIPTION

The principles, uses, and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout.

### Definitions

To facilitate an understanding of the present invention, a number of terms and phrases are defined below. It is to be understood that these terms and phrases are for the purpose of description and not of limitation, such that the terminology or phraseology of the present specification is to be interpreted by the skilled artisan in light of the teachings and guidance presented herein, in combination with the knowledge of one of ordinary skill in the art.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

The terms “subject”, “patient” or “individual” generally refer to a human, in particular, a female subject. In some embodiment, the tested subject is asymptomatic for ovarian cancer. In some embodiment, the tested subject is symptomatic for ovarian cancer. In some embodiment, the tested subject is at high risk for ovarian cancer (for example, having genetic disposition, such as PRCA positive).

According to some embodiments, the cancer that may be detected/predicted/determined is ovarian cancer. In some embodiments, the ovarian cancer may be ovarian carcinoma, Ovarian fibroma leomioma, Granulosa cell tumor, mucinous—type 1 ovarian carcinoma, Endometrioid adenocarcinoma, BRCA positive, ovarian cyst, neoplasia, papillary carcinoma of ovary, dermoid cyst, and the like. In some embodiments, the type of ovarian cancer that may be serous, endometrioid, mucinous, and clear cell tumors. In some embodiments, prediction/determination/identification of ovarian cancer may include early detection and/or prediction/determination/detection of a specific stage of the cancer.

As used herein, the term “vaginal secretion(s)” is directed to various discharges from the vagina, including, for example, fluids, cells, debris, mucus (such as vaginal mucus), fallopian secretions, and the like, or any combination thereof. In some embodiments, the vaginal secretion is not processed prior to being tested. In some embodiments, the vaginal secretions do not include menstrual fluids/blood. In some embodiments, the vaginal secretions may be collected by any suitable collection device, including, for example, a pad, a tampon, a swab, and the like. In some embodiments, a collection device may be a collection device as disclosed in International Patent Application Publication No. WO/2020/152679.

As used herein, the term “biomarker” is directed to a molecule (such as a peptide, protein, nucleic acid, and the like), which is present/expressed/produced/secreted by cells/tissues of the female subject and is capable of being detected (at least a measurable fragment thereof) in the vaginal secretion. In some embodiments, the biomarker is a peptide or protein involved in one or more cellular pathways. In some embodiments, the biomarker may be immune-related (i.e., a biomarker (such as, protein) involved in immunological pathways). In some embodiments, the biomarker may be cancer-related (i.e., a biomarker (such as, protein) involved in cancer-related pathways, in particular, ovarian cancer related pathways). In some embodiments, as further detailed herein, the presence and/or abundancy/concentration of one or more specific biomarkers (i.e., a combination of biomarkers) in the vaginal secretion are indicative of a disease (such as, cancer) condition and/or the stage of the disease. In some embodiments, as further detailed herein, the identification of the biomarker may be performed by any suitable means or reagents, such as, for example, an antibody (polyclonal, monoclonal, aptamers, etc.), directed against (capable of binding) a specific protein biomarker (or fragment thereof). In some embodiments, the reagents (such as antibodies) may include one or more tagging or otherwise identification moieties, facilitating their recognition and quantitation in downstream detection applications. In some embodiments, any type of immunoassay can be utilized for detection of biomarkers in vaginal secretion samples, including, for example, labeled antibodies, ELISA, bead-based methods (Luminex), and the like.

In some embodiments, such identification reagents (for example, specific antibodies) may be provided in the form of a test kit, optionally together with written instructions for performing the evaluation the level of one or more specific biomarkers in vaginal secretions, to determine/predict ovarian cancer (and/or or stage thereof) in a tested subject.

According to some embodiments, the methods and kits disclosed herein can be used for predicting the likelihood of ovarian cancer in a subject based on detecting/measuring the levels of one or more specific biomarker(s) in a vaginal secretion of the subject. As exemplified herein, changes/differences in the expression levels/abundancy/relative level/concentration of the one or more specific biomarker(s), as compared to a control group (for example, healthy female subjects/females not afflicted with ovarian cancer), are predictive or indicative of ovarian cancer in the tested subject and/or of the stage thereof. In some embodiments, the changes/differences are statistically significant.

As used herein, the term “treating” includes abrogating, substantially inhibiting, slowing or reversing the progression of ovarian cancer condition, substantially ameliorating clinical or aesthetical symptoms of the cancer or substantially preventing the appearance of clinical or other symptoms of the cancer.

According to some embodiments one or more specific biomarkers may be detected in vaginal secretions of a female subject to determine the presence and/or stage of a cancer condition. In some embodiments, at least one specific biomarker may be detected in vaginal secretions a female subject to determine the presence and/or stage of a cancer condition. In some embodiments, at least two specific biomarkers may be detected in vaginal secretions a female subject to determine the presence and/or stage of a cancer condition. In some embodiments, at least three specific biomarkers may be detected in vaginal secretions a female subject to determine the presence and/or stage of a cancer condition. In some embodiments, at least four specific biomarkers may be detected in vaginal secretions a female subject to determine the presence and/or stage of a cancer condition. In some embodiments, at least five specific biomarkers may be detected in vaginal secretions a female subject to determine the presence and/or stage of a cancer condition. In some embodiments, at least six specific biomarkers may be detected in vaginal secretions a female subject to determine the presence and/or stage of a cancer condition. In some embodiments, more than one specific biomarker may be detected in vaginal secretions a female subject to determine the presence and/or stage of a cancer condition. In some embodiments, more than two specific biomarkers may be detected in vaginal secretions a female subject to determine the presence and/or stage of a cancer condition. In some embodiments, more than three specific biomarkers may be detected in vaginal secretions a female subject to determine the presence and/or stage of a cancer condition. In some embodiments, more than four specific biomarkers may be detected in vaginal secretions a female subject to determine the presence and/or stage of a cancer condition. In some embodiments, more than five specific biomarkers may be detected in vaginal secretions a female subject to determine the presence and/or stage of a cancer condition. In some embodiments, more than six specific biomarkers may be detected in vaginal secretions a female subject to determine the presence and/or stage of a cancer condition. In some embodiments, a combination of two or more specific biomarkers may be detected in vaginal secretions a female subject to determine the presence and/or stage of a cancer condition. In some embodiments, a combination of at least two specific biomarkers may be detected in vaginal secretions a female subject to determine the presence and/or stage of a cancer condition.

According to some embodiments, specific biomarker(s) presence and/or concentration/abundancy may be determined/measured in vaginal secretions in healthy women and in women suffering from various conditions related to ovarian cancer.

In some embodiments, a combination of one or more biomarkers may be selected to be used as a reliable indicator of ovarian cancer and/or stage thereof.

According to some embodiments, one or more such specific biomarkers differences in expression levels, abundancy and/or relative expression level between healthy and sick/cancer afflicted patients, may each be used for some particular condition. A combination of such biomarkers may be sensitive to multiple conditions. In some embodiments, some biomarkers may produce false positives for certain individuals or conditions while other biomarkers do not show a false positive for those individuals or conditions. In some embodiments some biomarkers may produce false positives when used alone, but when used in combination with other biomarkers, may reduce false positive results and increase accuracy and specificity. According to some embodiments, the combination of biomarkers can thus provide an improved reliability measure in differentiating sick and healthy subjects and/or in differentiating between disease stages and/or early detection thereof.

In some embodiments, one or more specific biomarkers used in a combination of biomarkers may each be assigned the same or different weight, when predicting or indicating ovarian cancer in the tested subject and/or of the stage thereof.

In some embodiments, a score may be assigned to specific biomarkers with respect of their predictive/indicative value of cancer and/or stage thereof. In some embodiments, the score of a specific biomarker may change depending if used alone or in combination with one or more additional biomarkers. In some embodiments, the score may be a confidence score.

In some embodiments, one or more of the following biomarkers may be detected and/or measured in vaginal secretions, to determine the presence of cancer, in particular, ovarian cancer. In some embodiments, a combination of biomarkers (i.e., panel of biomarkers) may be detected and/or measured in the vaginal secretions. In some embodiments, a kit may be supplied for sampling and/or detecting and/or measuring quantities of the one or more biomarkers.

According to some embodiments, the biomarkers may be selected from, for example: AFP, Total PSA, TRAIL, CEA, SCF, OPN, b-HCG, TGFa, CA9, Midkine, TGM2, EpCAM, GALECTIN-1, GALECTIN-9, PD-L1, CD40 ligand, Flt-3 ligand, Fraktaline, IL-1a, IL-2, IL-8, IL-13, IL-33, IP-10, MIP-1a, MIP-1b, MIP-3b, and/or TNF-a. Each possibility is a separate embodiment.

According to some embodiments, a combination of biomarkers may include, for example, a combination of two or more biomarkers selected from: AFP, Total PSA, TRAIL, CEA, SCF, OPN, b-HCG, TGFa, CA9, Midkine, TGM2, EpCAM, GALECTIN-1, GALECTIN-9, PD-L1, CD40 ligand, Flt-3 ligand, Fraktaline, IL-1a, IL-2, IL-8, IL-13, IL-33, IP-10, MIP-1a, MIP-1b, MIP-3b, and/or TNF-a. Each possibility is a separate embodiment.

According to some embodiments, a combination of biomarkers may include, for example, a combination of two or more biomarkers selected from: AFP, Total PSA, MIF, TRAIL, Leptin, CEA, Ca-125, Prolactin, SCF, OPN, b-HCG, HE4, TGFa, CA9, Mesothelin, Midkine, TGM2, EpCAM, CD44, GALECTIN-1, GALECTIN-9, PD-L1, CD40 ligand, Flt-3 ligand, Fraktaline, IL-1a, IL-2, IL-8, IL-13, IL-33, IP-10, MIP-1a, MIP-1b, MIP-3b, TNF-a and/or VEGF. Each possibility is a separate embodiment.

According to some embodiments, the biomarkers may be selected from, for example: Total PSA, TRAIL, SCF, TGFa, Mesothelin, Midkine, TGM2, EpCAM, GALECTIN-1, GALECTIN-9, CD40 ligand, Flt-3 ligand, Fraktaline, IL-1a, IL-2, IL-13, IL-33, MIP-1a and/or MIP-1b. Each possibility is a separate embodiment.

According to some embodiments, a combination of biomarkers may include, for example, a combination of two or more biomarkers selected from: Total PSA, TRAIL, SCF, TGFa, Mesothelin, Midkine, TGM2, EpCAM, GALECTIN-1, GALECTIN-9, CD40 ligand, Flt-3 ligand, Fraktaline, IL-1a, IL-2, IL-13, IL-33, MIP-1a and/or MIP-1b. Each possibility is a separate embodiment.

According to some embodiments, the biomarkers may be selected from: TRAIL, TGFa, Midkine, TGM2, CD40 ligand, Flt-3 ligand, Fraktaline and/or PD-L1. Each possibility is a separate embodiment.

According to some embodiments, a combination of biomarkers may include, for example, a combination of two or more biomarkers selected from: TRAIL, TGFa, Midkine, TGM2, CD40 ligand, Flt-3 ligand, Fraktaline and/or PD-L1. Each possibility is a separate embodiment.

According to some embodiments, a combination of biomarkers may include, for example, at least one biomarker selected from: TRAIL, TGFa, Midkine, TGM2, CD40 ligand, CD-44, Flt-3 ligand, Fraktaline and/or PD-L1 and at least one additional biomarker selected from: Total PSA, MIF, Leptin, CEA, SCF, Mesothelin, EpCAM, GALECTIN-1, IL-1a, IL-2, IL-8, IL-13, IL-33, IP-10, MIP-1a, MIP-1b, MIP-3b, TNF-a, and/or VEGF. Each possibility is a separate embodiment.

According to some embodiments, a combination of biomarkers may include, for example, a combination of two or more biomarkers selected from: CD40 Ligand, CD44, CEA, Flt3 Ligand, Fraktaline, IL-8, MIP-1a, MIP-1b, MIP-3a, TGF-alpha, TGM2, Total PSA, TRAIL and/or VEGF. Each possibility is a separate embodiment.

According to some embodiments, a combination of biomarkers may include, for example, a combination of two or more biomarkers selected from: CD40 Ligand, CD44, CEA, Flt-3 Ligand, GALECTIN-9, IL-2, IP-10, Mesothelin, Midkine, MIF, MIP-1a, MIP-1b, OPN, PD-L1, SCF, TGF-alpha (also named herein TGF-a or TGF-a), TNF-alpha (also named herein TNF-a or TNF-α), TRAIL and/or VEGF. Each possibility is a separate embodiment.

According to some embodiments, a combination of biomarkers may include, for example, a combination of two or more biomarkers selected from: MIF, CD40 Ligand, CD44, CEA, Flt-3 Ligand, GALECTIN-9, Leptin, Midkine, MIP-1a, MIP-1b, MIP-3b, PD-L1, Prolactin, TGF-a, TGM2, TRAIL and/or VEGF. Each possibility is a separate embodiment.

According to some embodiments, a combination of biomarkers may include, for example, a combination of two or more biomarkers selected from: CD-40 Ligand, CA9, Midkine, TGF-a and/or Total PSA. Each possibility is a separate embodiment.

According to some embodiments, a combination of biomarkers may include, for example, a combination of two or more biomarkers selected from: CD-40 Ligand, CA9, CD44, MIP-1b and/or TRAIL. Each possibility is a separate embodiment.

According to some embodiments, a combination of biomarkers may include, for example, a combination of two or more biomarkers selected from: IL-2, CA9, CD44, MIP-1b and/or Total PSA. Each possibility is a separate embodiment.

According to some embodiments, a combination of biomarkers may include, for example, a combination of two or more biomarkers selected from: IL-2, CD-40 Ligand, CD44, MIP-1a and/or Leptin. Each possibility is a separate embodiment.

In some embodiments, a combination of specific biomarkers may include at least: Total PSA, CA9, CD44 and IL-2.

In some embodiments, a combination of specific biomarkers may include at least: Total PSA, CA9, MIP1b and IL-2.

In some embodiments, a combination of specific biomarkers may include at least: Total PSA, CA9, VEGF and IL-2.

In some embodiments, a combination of specific biomarkers may include at least: Total PSA, MIP1b, CD44 and IL-2.

In some embodiments, a combination of specific biomarkers may include at least: Total PSA, EGF, MIP1b and IL-2.

In some embodiments, a combination of specific biomarkers may include at least: Total PSA, EPCAM, MIP-1b and IL-2.

In some embodiments, a combination of specific biomarkers may include at least: Total PSA, IL-1a, MIP-1b and IL-2.

In some embodiments, a combination of specific biomarkers may include at least: Total PSA, CA9, IL-1a and MIP-1b.

In some embodiments, a combination of specific biomarkers may include at least: Total PSA, VEGF, MIP-1b and IL-2.

In some embodiments, a combination of specific biomarkers may include at least: Total PSA, CAE, CA9 and MIP-1b.

In some embodiments, a combination of specific biomarkers may include at least: Total PSA, CA9 and IL-2.

In some embodiments, a combination of specific biomarkers may include at least: Total PSA, MIP-1b and IL-2.

In some embodiments, a combination of specific biomarkers may include at least: Total PSA, CD44 and IL-2.

In some embodiments, a combination of specific biomarkers may include at least: Total PSA, EGF and MIP-1b.

In some embodiments, a combination of specific biomarkers may include at least: Total PSA, CA9 and MIP-1b.

In some embodiments, a combination of specific biomarkers may include at least: Total PSA, CEA and MIP-1b.

In some embodiments, a combination of specific biomarkers may include at least: Total PSA, Midkine and MIP-1b.

In some embodiments, a combination of specific biomarkers may include at least: Total PSA, VEGF and IL-2.

In some embodiments, a combination of specific biomarkers may include at least: Total PSA, IL-33 and MIP-1b.

In some embodiments, a combination of specific biomarkers may include at least: Total PSA, VEGF and MIP-1b.

In some embodiments, a combination of specific biomarkers may include at least: Total PSA and MIP-1b.

In some embodiments, a combination of specific biomarkers may include at least: Total PSA and IL-2.

In some embodiments, a combination of specific biomarkers may include at least: Total PSA and CA9.

In some embodiments, a combination of specific biomarkers may include at least: TGF-a and CD-40 ligand.

In some embodiments, a combination of specific biomarkers may include at least: MIP-3a and MIP-1b.

In some embodiments, a combination of specific biomarkers may include at least: VEGF and CD-40 Ligand.

In some embodiments, a combination of specific biomarkers may include at least: CD-44 and CD-40 Ligand.

In some embodiments, a combination of specific biomarkers may include at least: Total PSA and AFP.

In some embodiments, a combination of specific biomarkers may include at least: Total PSA and b-HCG.

In some embodiments, a combination of specific biomarkers may include at least: Total PSA and IL-8.

According to some embodiments the biomarkers may be selected from at least one of: Interleukin 2 (IL-2), IL-1a, Interleukin 13 (IL-13), CD-40 Ligand, Flt-3 Ligand, Fraktaline, IL-33, GALECTIN-9, Midkine, EpCAM, MIP 1a, MIP-1b, MIP-3a, MIP-3b, PD-L1, RANTES, TGF-alpha, TGM2, Total PSA, Eotoxin, FGF-basic, G-CSF, GM-CSF, Granzyme B, Gro-a, Gro-b, IFN-a, IFN-b, CTLA4, PD-1, IL-1b, IL-1ra, IL-3, IL-4, IL-5, IL-10, IL-15, IL-17A, IL-17E, MCP-1, PDGF-AA, PDGF-AB BB and/or TRAIL, Each possibility is a separate embodiment.

According to some embodiments the biomarkers may be selected from at least one of: CD40 ligand, Flt-3 Ligand, Fraktaline, GALECTIN-9, Midkine, EpCAM, IL-1a, IL-2, IL-13, IL-33, MIP-1b, MIP-3b TGF-alpha, TGM2 and/or TRAIL.

According to some embodiments, at least one specific biomarker may be IL-2. In some embodiments, at least one specific biomarker may be IL-13. In some embodiments, at least one specific biomarker may be TRAIL. In some embodiments, at least one specific biomarker may be PSA (Total PSA). In some embodiments, at least one specific biomarker may be SCF. In some embodiments, at least one specific biomarker may be TGFa. In some embodiments, at least one specific biomarker may be Midkine. In some embodiments, at least one specific biomarker may be TGM2. In some embodiments, at least one specific biomarker may be EpCAM. In some embodiments, at least one specific biomarker may be GALECTIN-1. In some embodiments, at least one specific biomarker may be GALECTIN-9. In some embodiments, at least one specific biomarker may be CD40 ligand. In some embodiments, at least one specific biomarker may be Flt-3 ligand. In some embodiments, at least one specific biomarker may be Fraktaline. In some embodiments, at least one specific biomarker may be TGFa. IL-1a. In some embodiments, at least one specific biomarker may be IL-33. In some embodiments, at least one specific biomarker may be MIP-1a. In some embodiments, at least one specific biomarker may be MIP-1b.

In some embodiments the test for cancer (e.g. ovarian cancer) as disclosed herein may measure and/or detect a combination of one, two or more, three or more, four or more, five or more specific biomarkers. In some embodiments, the test may optionally be embodied in a kit.

According to some embodiments, specific biomarkers and/or combinations thereof, may be used to differentiate between grades of cancer (stages of cancer), differentiate between early stage and late stage cancer, differentiate between benign condition and early stage cancer, differentiate between benign condition and late stage cancer, and the like, or any combination thereof. In some embodiments, specific biomarkers and/or combinations thereof, may be used to differentiate between low and High Grade epithelial ovarian cancers (EOC) and/or low grade EOC and/or benign conditions.

According to some embodiments, such specific biomarkers (identification of which can be used to differentiate between low and High Grade epithelial ovarian cancers (EOC) and/or low grade EOC and/or benign conditions) may be selected from one or more of: CD40 ligand, EGF, Eotaxin, FGF basic, Flt-3 ligand, Fraktaline, G-CSF, GM-CSF, Granzyme B, Gro-a, Gro-B, IFN-a, IFN-b, IFN-gamma, IL-1a, IL-1b, IL-1ra, IL-2, IL-3, IL-4, IL-5, IL-6, IL-7, IL-8, IL-10, IL-12, IL-13, IL-15, IL-17A, IL-17E, IL-33, IP-10, IP70, MCP-1, MIP-1a, MIP-1b, MIP-3a, MIP-3b, PDGF-AA, PDGF-AB BB, PD-L1, RANTES, TGF-a, TNF-a, TRAIL and/or VEGF. Each possibility is a separate embodiment.

According to some embodiments, such specific biomarkers (identification of which can be used to differentiate between low and High Grade epithelial ovarian cancers (EOC) and/or low grade EOC and/or benign conditions) may be selected from one or more of: CD44, IL-8, PD-L1, IL-2, MIP-1b, CD-40 Ligand, Flt3-Ligand, TRAIL, TGF-a, IL-13, IL-33 and/or fractalkine. Each possibility is a separate embodiment.

According to some embodiments, such biomarkers (differentiating between low and High Grade epithelial ovarian cancers (EOC) and/or low grade EOC and/or benign conditions) may be immunological proteins, or proteins related to the immunology system.

According to some embodiments, specific biomarkers detected in vaginal secretions may discriminate between high stage EOC, low stage EOC and/or benign gynecological conditions. In some embodiments, low grade EOC vaginal secretions may contain significantly higher levels of: CD40 ligand, Flt-3 ligand, IL-2, IL-33, MIP-1b, MIP-3a, MIP-3b, PD-L1, RANTES, TGF-a and/or TRAIL compared to benign gynecological conditions. Such specific biomarkers (and/or combinations thereof) may be used and/or be part of a kit for differentiating high grade EOC low grade EOC and/or benign conditions.

According to some embodiments, the biomarker is not CA125. In some embodiments, a combination of biomarkers does not include CA125.

According to some embodiments, in some instances, the use/determination of one specific biomarker may not by itself be sufficiently indicative of cancer and/or grade thereof and additional biomarkers (i.e., combination of biomarkers) may be utilized to increase accuracy and specificity.

According to some embodiments, in some instances, the determination of one “positive” biomarker (i.e. based on the levels thereof in vaginal secretion) is not by itself indicative of cancer and additional one or more biomarkers (for example, at least two, at lest three biomarkers) may be used to substantiate the results and thereby reduce false positive results.

According to some embodiments, in some instances, a recommendation to test/check for additional biomarkers (and/or other suitable tests) may be provided based on the measured/detected levels of a specific biomarkers in vaginal secretions.

According to some embodiments, the significance of a specific biomarker as being indicative of cancer and/or grade thereof may be below a threshold when tested alone, and the significance thereof may increase when used in combination with one or more specific biomarkers.

According to some embodiments, when used in combination, different biomarkers may be assigned different weight, depending, for example, on the type of the biomarker, degree of deviation in its levels, confidence score thereof, and the like, when determining cancer condition and/or stage thereof.

According to some embodiments, there is provided a kit for detection of ovarian cancer in a sample obtained from vaginal secretion of a subject, the kit includes one or more reagents capable of detecting the level of one or more specific biomarkers.

According to some embodiments, the methods and kits disclosed herein can further be utilized to assess/determine treatment efficacy (therapeutic efficiency) of various types of cancer treatments. According to some embodiments, the expression/relative expression levels of the specific biomarker or combination of biomarkers in vaginal secretion are determined prior to commencement of treatment, during and/or after treatment at desired time points. In some embodiments, change(s) in the expression/relative expression of such biomarkers is indicative of treatment efficacy.

In some embodiments, the methods and kits disclosed herein can be used for determining if the subject is developing cancer. The relative levels of expression of the specific biomarker(s) are determined over a period of time, whereby changes in the biomarker(s) expression are indicative as to the progression of the cancer development.

According to some embodiments, the methods and kits disclosed herein may further be utilized to provide a treatment recommendation. Based on the levels of the determined biomarker or combination of biomarkers, a suitable treatment regime may be provided. Such treatment regime, may include, for recommendation regarding chemotherapy, immunotherapy, radiotherapy, surgical therapy, and the like. In some embodiments, a recommendation(s) may be to use one or more specific types of chemotherapeutic or immunotherapeutic agent, such as, but not limited to: PARP inhibitors, Olaparib, Rucaparib, Niraparib, Talazoparib, cisplatin, carboplatin, paclitaxel, docetaxel, and the like, or any combination thereof.

According to some embodiments, the specific biomarkers and combinations thereof disclosed herein provide sensitive, specific and accurate methods for predicting the presence of or detecting ovarian cancer at various stages of its progression. The evaluation of samples as described may also correlate with the presence of a pre-malignant or a pre-clinical condition in a patient.

According to some embodiments, provided herein are methods for identifying and characterizing specific biomarkers suitable for use in detecting early-stage ovarian cancer.

According to some embodiments, the methods and kits disclosed herein are accurate, specific and sensitive with respect of detecting/predicting and or determining ovarian cancer condition and/or stage thereof.

The following abbreviations of biomarkers are used herein:
CEA carcinoembryonic antigen
CA9 Carbonic anhydrase IX
Flt-3 Ligand Fms-like tyrosine kinase 3 ligand
IL-2 Interleukin 2
IL-8 Interleukin 8
IP-10 Interferon (IFN)-γ inducible protein 10
MIF Macrophage migration inhibitory factor
MIP-1a macrophage inflammatory protein 1-alpha
MIP-1b Macrophage Inflammatory Protein-1 beta
MIP-3a Macrophage inflammatory protein-3 alpha
OPN Osteopontin
PD-L1 Programmed death-ligand 1
SCF Stem cell factor
TGF-a Transforming growth factor alpha
TGM2 Transglutaminase 2
TNF-a Tumor necrosis factor alpha
Total PSA Total Prostate Specific Antigen
TRAIL TNF-related apoptosis-inducing ligand
VEGF Vascular endothelial growth factor

In the description and claims of the application, the words “include” and “have”, and forms thereof, are not limited to members in a list with which the words may be associated. As used herein, the term comprising includes the term consisting of.

As used herein, the term “about” may be used to specify a value of a quantity or parameter (e.g. the length of an element) to within a continuous range of values in the neighborhood of (and including) a given (stated) value. According to some embodiments, “about” may specify the value of a parameter to be between 80% and 120% of the given value.

As used herein, according to some embodiments, the terms “substantially” and “about” may be interchangeable.

In the description and claims of the application, the words “include” and “have”, and forms thereof, are not limited to members in a list with which the words may be associated. As used herein, the term comprising includes the term consisting of.

While a number of exemplary aspects and embodiments have been discussed above, those of skill in the art will recognize certain modifications, permutations, additions and sub-combinations thereof. It is therefore intended that the following appended claims and claims hereafter introduced be interpreted to include all such modifications, permutations, additions and sub-combinations as are within their true spirit and scope.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

### Methods

### Sample Collection

The following biological samples were collected:

Vaginal secretion samples. Vaginal fluids were collected during surgical procedure or during routine physical examination using specific sterile applicator/cotton swab, and pretreated to maintain proteins. Samples were frozen immediately after collection at −80 until analysis. Menstrual blood or pathologic uterine bleeding may interfere with the biomarker analysis; therefore, samples are taken from menstruating or bleeding women.

Blood: On the day of vaginal fluids collection 5 ml of peripheral blood were drawn from each volunteer. Immediately after blood collection the tubes were centrifuged at 4 C 1800 rpm for 15 minutes. Serum was aliquoted into 2 similar Eppendorf tubes and frozen at −80 until analysis.

### Biomarkers Analysis:

In order to perform quantitation of multiple biomarkers in vaginal secretions and in blood, Bead based immunodetection (Luminex) assay was performed.

Briefly, sample is added to a mixture of color-coded beads, pre-coated with analyte-specific capture antibodies. Plate is incubated 1 hour with shaking. Next, biotinylated detection antibodies specific to the analytes of interest are added and form an antibody-antigen sandwich. Phycoerythrin (PE)-conjugated streptavidin is added. It binds to the biotinylated detection antibodies.

Beads were read on a dual-laser flow-based detection instrument, BioPlex 2000.

Analysis was performed using BioRad software, and GraphPad Prism.

For each biomarker, a specific calibration curve was generated. To calculate the concentration of each biomarker in a specimen, the value from the specimen was interpolated from the calibration curve.

Where indicated, biomarker concentrations were compared between healthy subjects and adult patients diagnosed with various conditions related to ovarian cancer, including, for example, ovarian carcinoma, Ovarian fibroma leomioma, Granulosa cell tumor, mucinous—type 1 ovarian carcinoma, Endometrioid adenocarcinoma, BRCA positive, ovarian cyst, neoplasia, papillary carcinoma of ovary and dermoid cyst.

The ability of various biomarkers to differentiate between healthy and sick patents can be seen in the results below. For example, those biomarkers where a significant number (e.g. at least 2 and/or 5 and/or 8 and/or 10) of the sick patients (represented by dots above “Patient”) are out of the variance range of the healthy controls (represented by the boxes above “Healthy”) may be used to identify sick patients. For example, those biomarkers where a significant number (e.g. at least 2 and/or 5 and/or 8 and/or 10 and/or all) of the healthy patients (represented by dots above “Healthy”) are out of the variance range of the sick patients (represented by the boxes above “Patients”) may be used to identify healthy patients. For example, those biomarkers with a low “P” value (e.g. less than 0.5 and/or 0.2 and/or 0.1 and/or 0.05 and/or 0.01 and/or 0.005) may be used to differentiate healthy individuals from sick patients.

### Example 1—Determining Specific Biomarker Levels in Blood Samples and Vaginal Secretions Samples Obtained from Healthy Subjects and Ovary Cancer Patients

The main goal of the study was to identify and characterize exemplary specific biomarkers suitable for use in detecting early-stage ovarian cancer.

Interim goal was to identify and characterize CA125 levels in vaginal secretions taken from patients diagnosed with ovarian cancer, and compare them to their levels in serum.

Characterization of biomarkers, namely IL-2 and IL-13 in vaginal secretions, collected from patients diagnosed with ovarian cancer, compared with secretions collected from vagina of healthy women.

### Sampling:

Vaginal discharge and blood samples were collected from 20 healthy women with benign gynecological conditions and from 22 women with advanced ovarian cancer, at Rambam Medical Center. All the subjects signed an informed consent form.

Vaginal discharge was taken from 22 patients diagnosed with advanced ovarian cancer. Of these, 10 patients were diagnosed with advanced high grade serous papillary carcinoma and received treatment (neoadjuvant) mean age—53.75, and 12 patients before surgery which did not receive still treatment (non-neoadjuvant) mean age—66.5.

Of the 12 patients, nine were diagnosed with high-grade serous papillary carcinoma, one was diagnosed with mucinous carcinoma and two were diagnosed with endometrioid adenocarcinoma.

CA125 levels were tested in serum and vaginal secretion samples. Other immunological biological markers (IL-2 and IL-13) were tested only in samples of vaginal discharge (18 healthy and 16 ovarian cancer patients).

### Experimental Methods

Serum preparation: Blood collected in tubes containing EDTA, centrifuged at 1800 rpm for 15 min at 4° C. Serum was collected and stored at −80° C.

Preparation of vaginal secretion: The vaginal fluid is collected during the operation using an applicator swab (cytobrush) and inserted into 15 ml test tube with 400 μl PBS. If not processed immediately, samples were frozen at −80° C.

The test tubes with the secretions were mixed in the vortex (for 30 seconds).

Centrifuged at 1800 rpm for 15 minutes at 4° C., supernatant collected, aliquoted and stored at −80° C.

CA125 levels in vaginal secretions and serum were measured using CA125/MUC16 ELISA—Human—Catalog Number: DY5609; Lot no: P235973, P253860 R&D Systems and DuoSet Axillary Reagent Catalog Number: DY008; R&D systems P245039, P256303. The experiment was performed according to the protocol.

HE4 vaginal secretions levels were measured using MILLIPLEX MAP Human Circulating Cancer Biomarker Magnetic Bead Panell Catalog Number: HCCBP1MAG-58K Lot no: 3451008 Power: Millipore. The experiment performed according to protocol. The measurements done using the Luminex200 analysis instrument system.

IL-2 and IL-13 levels in vaginal secretions were measured using a panel of Human XL Cytokine, Catalog Number LKTM014; lot number—P249764. Supplier: R&D. The experiment was performed according to a protocol. The measurements done using the Luminex200 analysis instrument system.

### Statistical Analysis:

Statistical calculations were made with the Mann-Whitney test in the Graph-Pad Prism software. The Mann-Whitney test is used to compare differences between two independent groups when the dependent variable is regular or continuous, but without a normal distribution.

### Results

### Comparison of CA125 Levels in Vaginal Secretions and Serum Levels

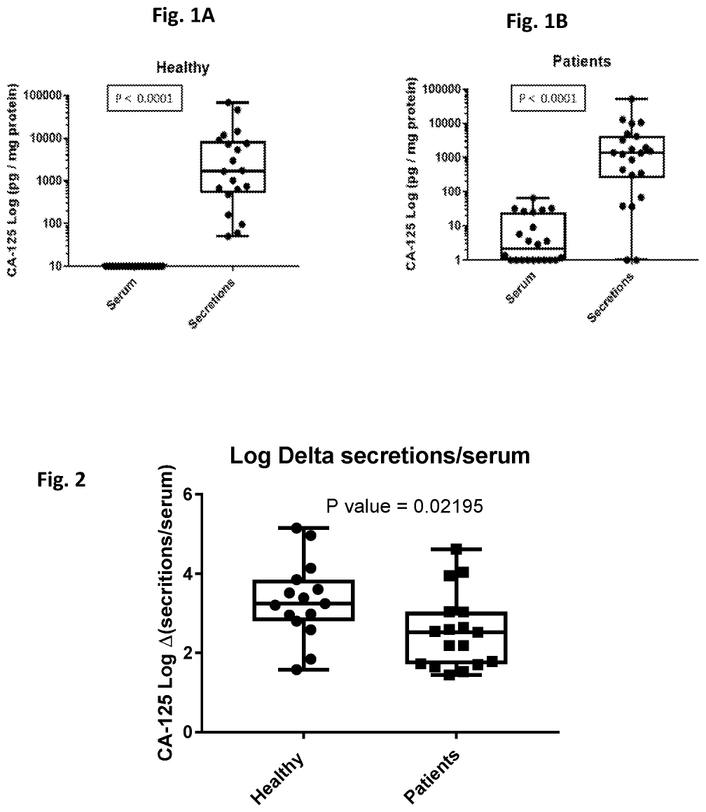

As seen from FIGS. 1A-B, it was found that CA125 levels were significantly higher in vaginal secretions than in serum in healthy women as well in ovarian cancer patients (regardless of treatment) (p<0.0001).

### The Difference (Δ) in CA125 Concentration Between Vaginal Secretions and Serum in Each Patient and Each Healthy

The difference between the group of patients and the group of healthy was obtained by dividing the concentration of CA125 in vaginal secretions by its concentration in the serum and applying log. As can be seen in FIG. 2 and Table 1 below, variance in the group of patients (regardless of treatment) was slightly lower than in the group of healthy, and the difference between the group of ovarian cancer patients and healthy was significant (P=0.022).

TABLE 1

| | Patients | Healthy |
|---|---|---|
| Var P | 0.838 | 0.893 |
| Stdev | 0.943 | 0.978 |

Testing of CA125 Serum Levels of the 3 Groups: Healthy Women, Sick Women after Neo-Adjuvant Treatment, Sick Women without Treatment (Non-Neoadjuvant)

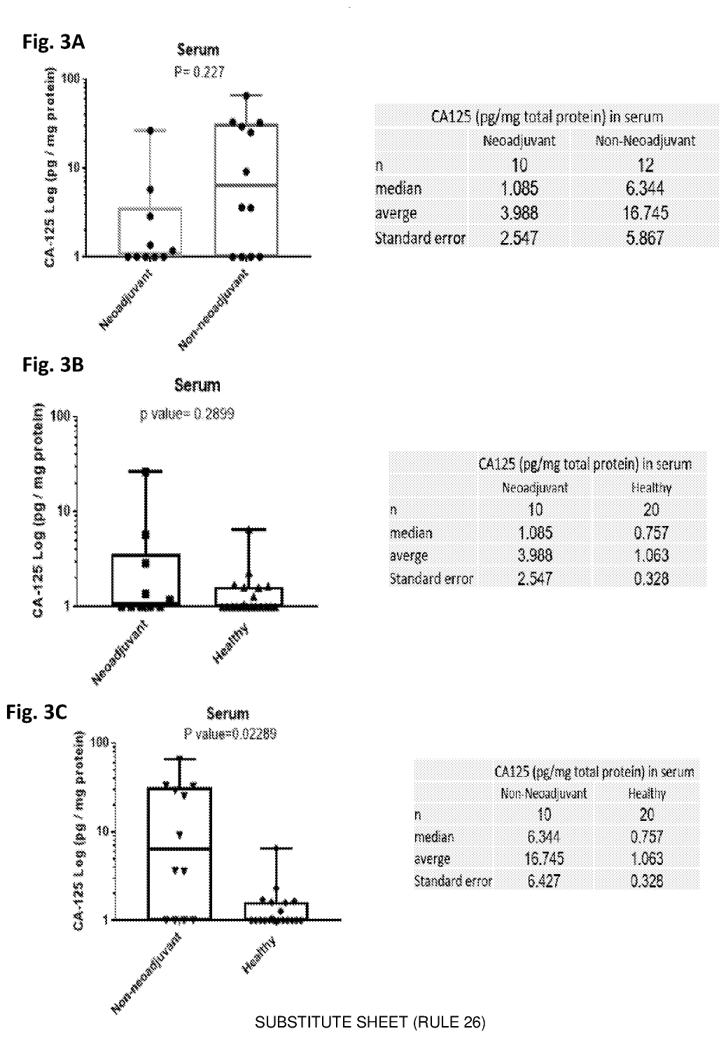

As shown in FIGS. 3A-C, no significant difference was found between the two groups of patients (P=0.227): CA125 levels in the serum of a group of ovarian cancer patients who received chemotherapy before surgery (neoadjuvant) and in women who did not receive chemotherapy before surgery (non-neoadjuvant).

In addition, no significant difference was found in CA125 serum levels of patients who received treatment before surgery (neoadjuvant) and healthy women (P=0.29).

Testing CA125 Levels in Vaginal Secretions of the 3 Groups: Healthy Women, Sick Women after Neo-Adjuvant Treatment, Sick Women without Treatment (Non-Neoadjuvant)

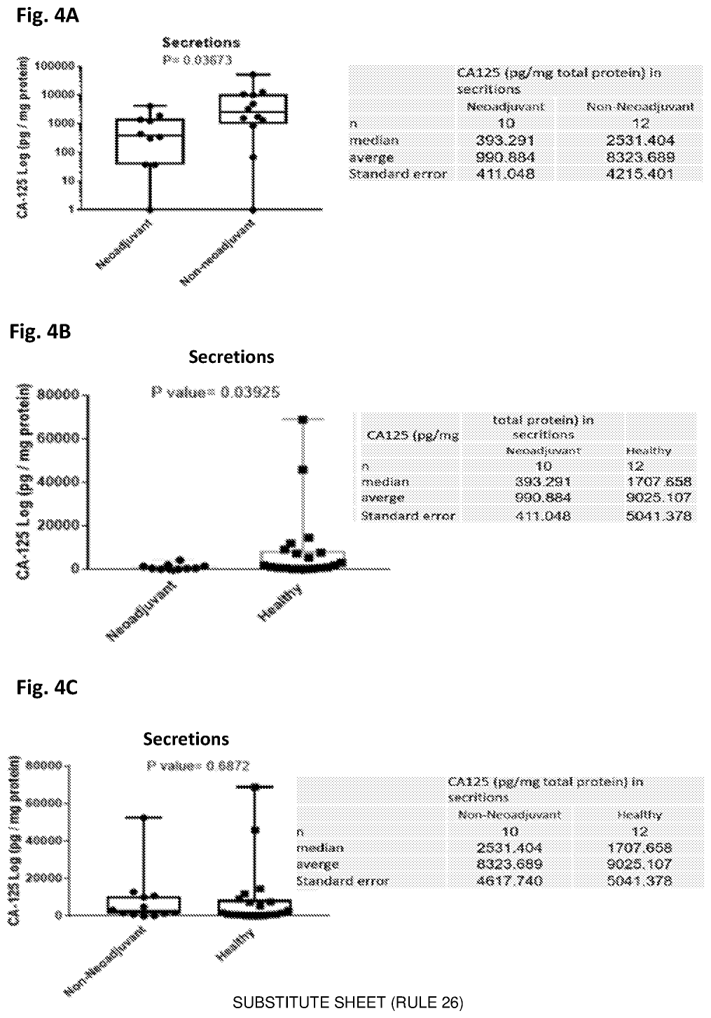

As demonstrated in FIGS. 4A-C, significantly lower CA125 levels were found in vaginal secretions in ovarian cancer patients who received chemotherapy before surgery (neoadjuvant) as compared to women who did not receive chemotherapy before surgery (non-neoadjuvant) (P=0.0367).

The difference (Δ) in CA125 levels between vaginal secretions and serum in patients who received treatment (neoadjuvant) and patients who did not receive treatment (non-neoadjuvant)

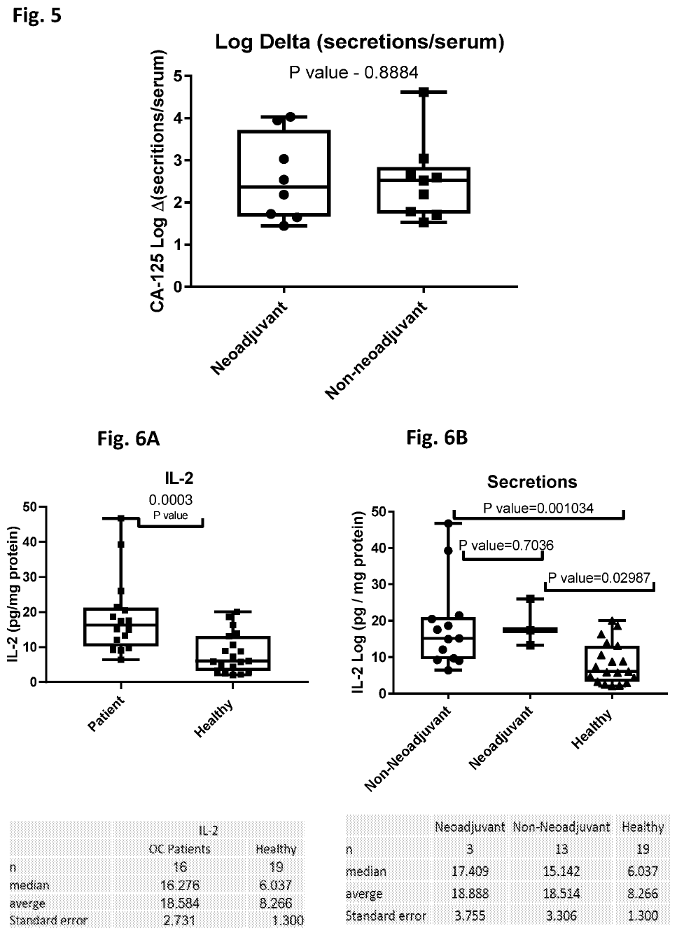

Examination of the difference between vaginal secretions and serum per participant (Δ) log (vaginal secretions/serum) demonstrated that the variance in the group of patients who received treatment before surgery (neoadjuvant) is lower than in non-neoadjuvant group (FIG. 5). No statistically significant difference in CA125 levels as log (vaginal secretions/serum) was found between the neoadjuvant/non-neoadjuvant groups (P=0.8884).

These results emphasize the unreliability of CA125 as a marker, whether obtained from serum or from vaginal secretions.

### Examination of IL-2 Levels in Vaginal Secretions

IL-2 levels were tested on 19 healthy women and 16 ovarian cancer patients, of whom only 3 received neoadjuvant therapy and 13 patients did not receive neoadjuvant treatment (non-neoadjuvant).

As shown in the results presented in FIG. 6A, IL-2 levels in vaginal secretions of all the patients were significantly higher, as compared to the values of healthy women, P=0.0003. so were the IL-2 levels in vaginal secretions of patients who received treatment (neoadjuvant) as compared to healthy women P=0.02987 (FIG. 6B).

IL-2 levels of patients who received neoadjuvant therapy were not statistically significant different from those of patients who did not receive treatment (non-neoadjuvant) (non-neoadjuvant), P=0.7036. (FIG. 6B).

Thus, IL-2 levels in vaginal secretions of ovarian cancer patients were statistically significantly higher than IL-2 levels in vaginal secretions of healthy women, P value=0.0003. IL-2 in vaginal secretions of ovarian cancer patients which received Neoadjuvant treatment were not statistically significantly different higher than IL-2 levels in vaginal secretions of ovarian cancer patients which not received neoadjuvant, P value=0.7036. IL-2 levels of neoadjuvant were statistically significantly different higher than IL-2 levels in vaginal secretions of healthy women P value=0.02987. IL-2 levels in vaginal secretions of ovarian cancer patients which not received neoadjuvant treatment statistically significantly higher than IL-2 levels in vaginal secretions of healthy women P value=0.001

### Examination of IL-13 Levels in Vaginal Secretions

IL-13 levels were tested on 19 healthy women and 16 ovarian cancer patients, of whom only 3 received neoadjuvant therapy and 13 did not receive neoadjuvant treatment (non-neoadjuvant).

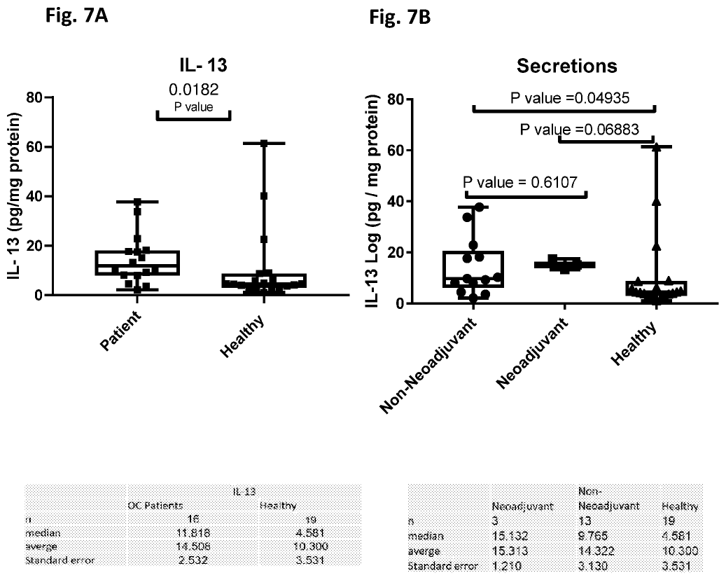

As shown in the results presented in FIGS. 7A-B, IL-13 levels in vaginal secretions of all patients were significantly higher than those of healthy women P=0.0182.

IL-13 levels in patients who received treatment (neoadjuvant) were not statistically significant different from those of patients who did not receive treatment (non-neoadjuvant), P=0.6107.

Thus, IL-13 levels in vaginal secretions of ovarian cancer patients statistically significantly higher than IL-13 levels in vaginal secretions of healthy women, P value=0.018.IL-13 in vaginal secretions of ovarian cancer patients which received Neoadjuvant treatment were not statistically significantly different higher than IL-13 levels in vaginal secretions of ovarian cancer patients which not received neoadjuvant, P value=0.6107. IL-13 levels of neoadjuvant are not statistically significantly different higher than IL-13 levels in vaginal secretions of healthy women P value=0.0688. IL-13 levels in vaginal secretions of ovarian cancer patients which not received neoadjuvant treatment statistically significantly higher than IL-13 levels in vaginal secretions of healthy women P value=0.04935.

### CONCLUSIONS

In the example, the levels of CA125 were compared in vaginal secretions of ovarian cancer patients and healthy women to test whether CA125 in vaginal secretions can serve as a diagnostic biological marker of early-stage ovarian cancer. The results showed that CA125 levels were significantly higher in vaginal secretions than in serum in both groups: in the ovarian cancer patients' group and in the control group (which included samples from healthy women).

Since CA125 is known as an unstable marker in the blood, which can elevate in various patient conditions, the problem may exist since the marker levels in the blood were also increased in some healthy women who came with benign gynecological conditions.

The results correspond to those made for serum levels indicating that about 25% of women suffer from ovarian cancer Stage 1 have a normal CA125 level while high CA125 level at times are found to be high among women with multiple benign conditions.

Surprisingly, it was found that IL-2 and IL-13 levels in vaginal secretions of ovarian cancer patients were significantly higher than IL-2 and IL-13 levels in the vaginal secretions of healthy women, indicating it may serve as a good predictor of early-stages disease.

The levels of the two biomarkers were similar in the group of patients who received treatment and the group of patients who did not receive treatment.

The results further indicate that measuring the levels of these biomarkers in vaginal secretions obviate the need to take blood samples and allow for the use of a home test kit that is easier and more accessible to any woman, thus facilitate routine testing. This is of particular importance for early detection of ovarian cancer having few if any clinal symptoms.

### Example 2—Determining Specific Biomarker Levels in Vaginal Secretions Samples Obtained from Healthy Subjects and Ovary Cancer Patients

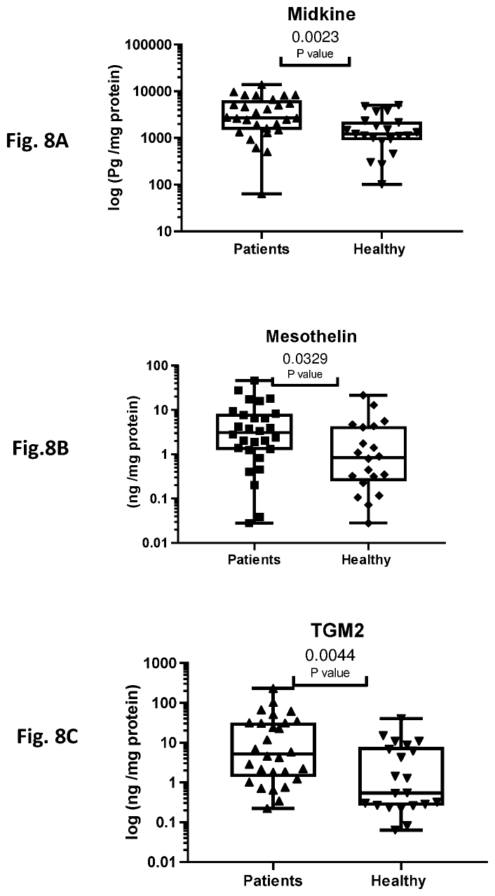

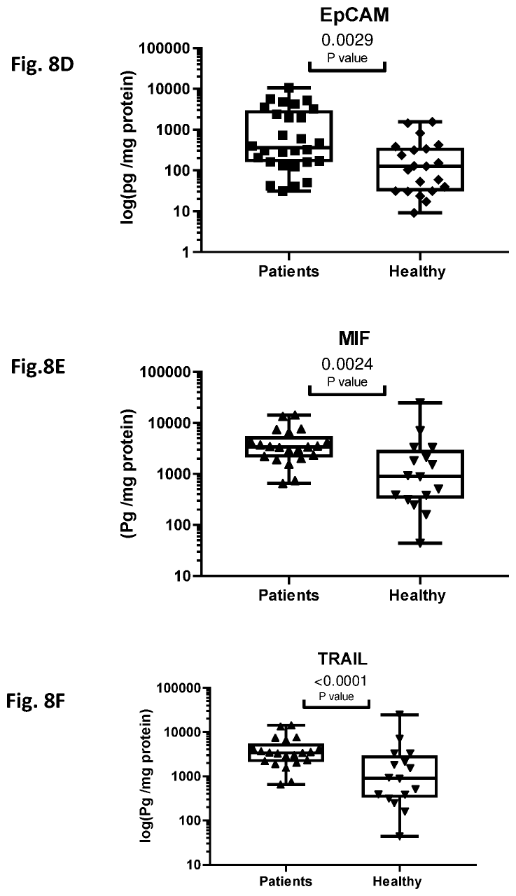

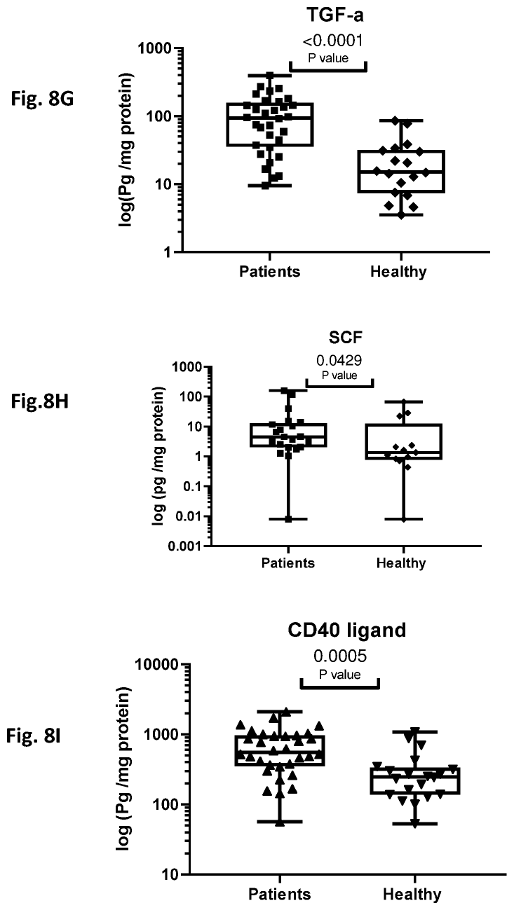

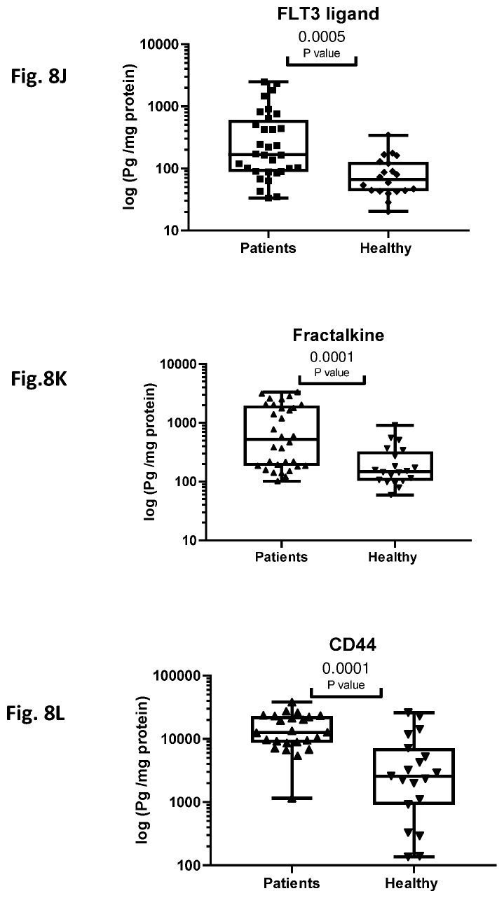

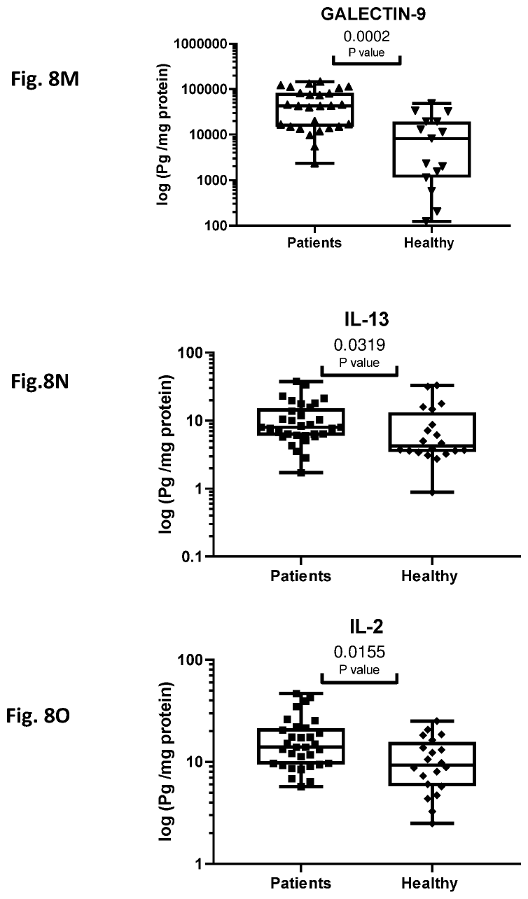

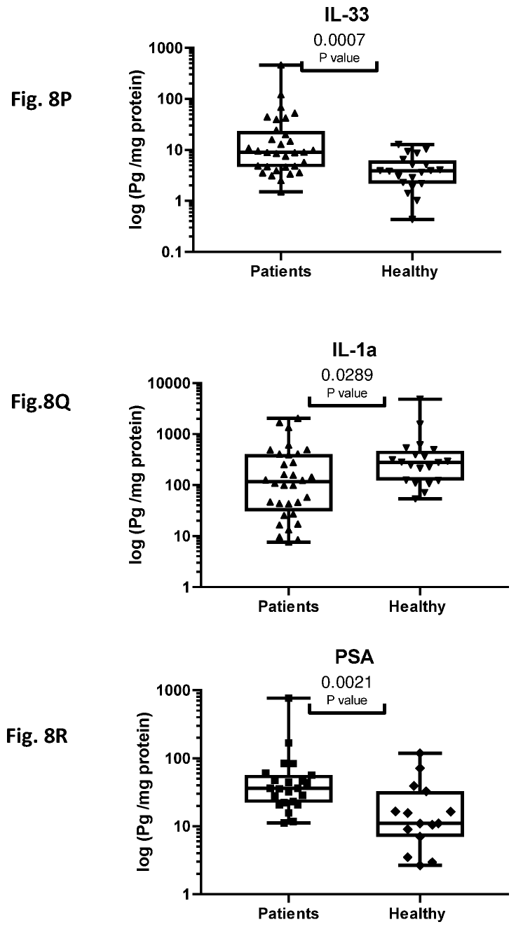

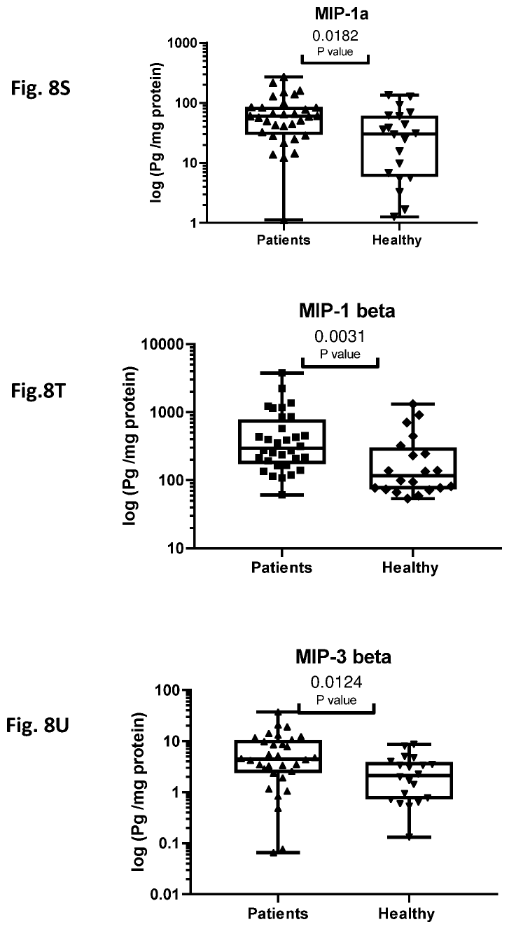

As detailed above, vaginal secretions from healthy subjects and from ovarian cancer patients were obtained, and the levels of various biomarkers were determined. The results are presented in FIGS. 8A-U, each presenting the relative expression levels of the respective biomarkers in healthy, and cancer afflicted subjects.

The following biomarkers were analyzed: FIG. 8A—Midkine, FIG. 8B—mesothelin; FIG. 8C—TGM2; FIG. 8D—EpCAM; FIG. 8E—MIF; FIG. 8F—TRAIL; FIG. 8G—TGF-a; FIG. 8H—SCF; FIG. 8I—CD40 Ligand; FIG. 8J—Flt3-Ligand; FIG. 8K—Fractalkine; FIG. 8L—CD44; FIG. 8M—GELACTINE 9; FIG. 8N—IL-13; FIG. 8O—IL-2; FIG. 8P—IL-33; FIG. 8Q—IL1a; FIG. 8R—PSA; FIG. 8S—MP-1; FIG. 8T—; MIP-1b; FIG. 8U—MIP-3b;

The results indicate that a statistically significant difference in the levels of the indicated biomarkers is observed between healthy and ovarian cancer patients.

In order to increase accuracy of detection/prediction, a combination of two or more biomarkers is detected in each sample.

### Example 3—Determining Specific Immune-Related Biomarker Levels in Vaginal Secretions Samples Obtained from High Stage Epithelial Ovarian Cancer (EOC) Patients, Benign Gynecological Conditions Subjects and Low Grade EOC Patient

Vaginal secretions from healthy subjects (n=18), from high stage epithelial ovarian cancer (EOC) patients (n=18) and from low grade EOC patient were obtained.

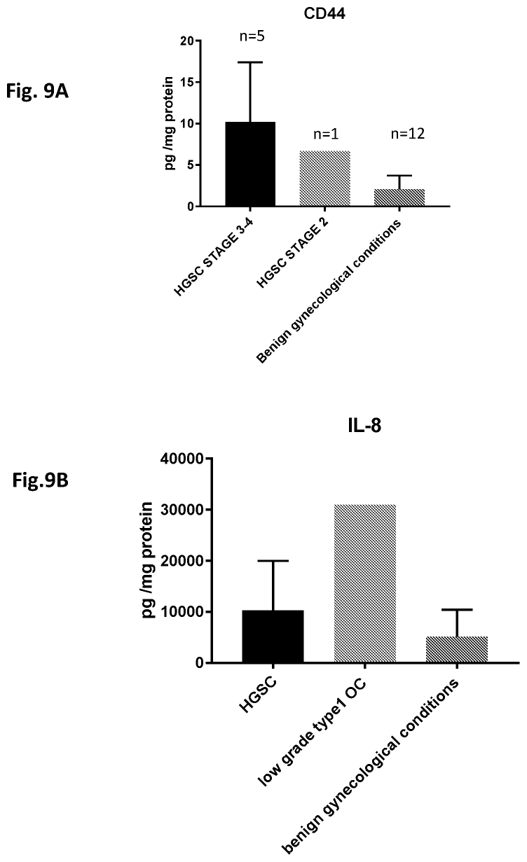

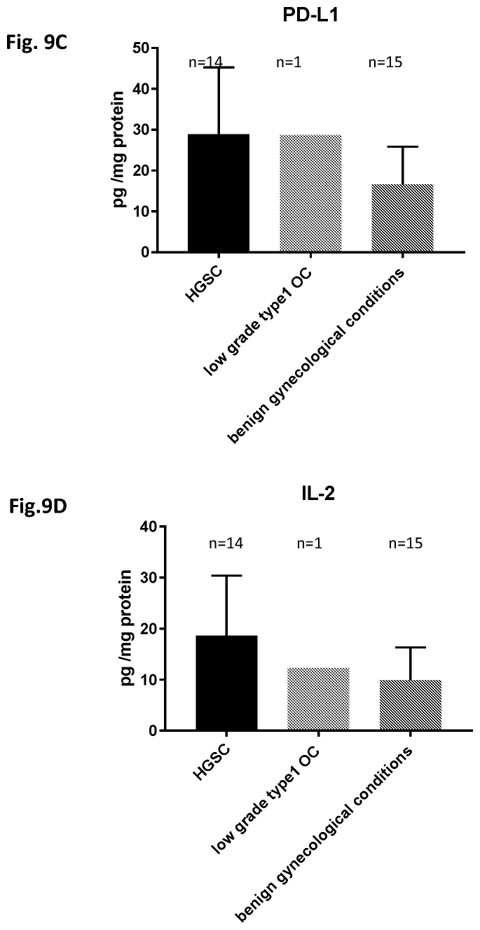

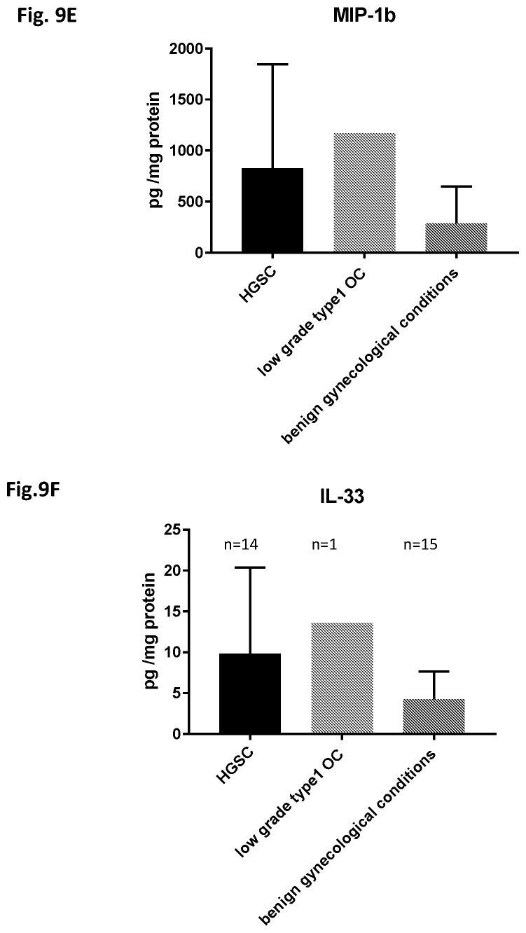

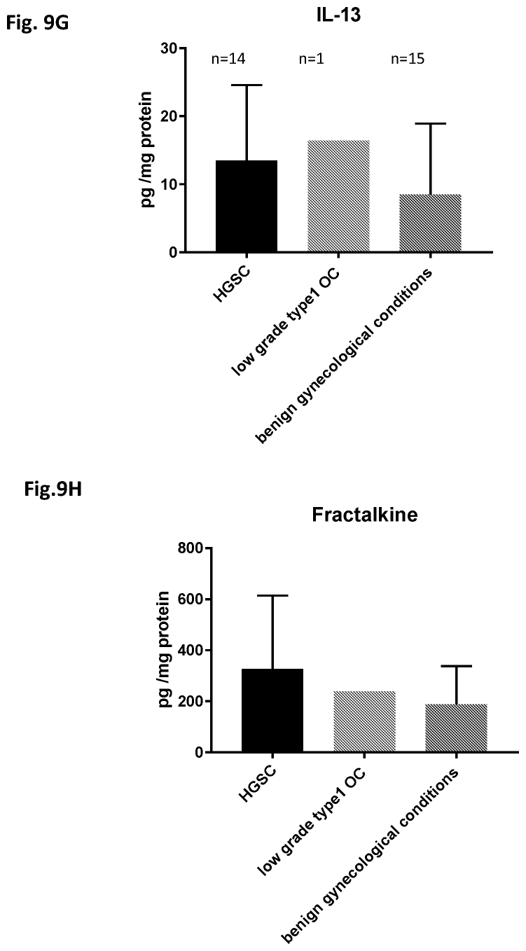

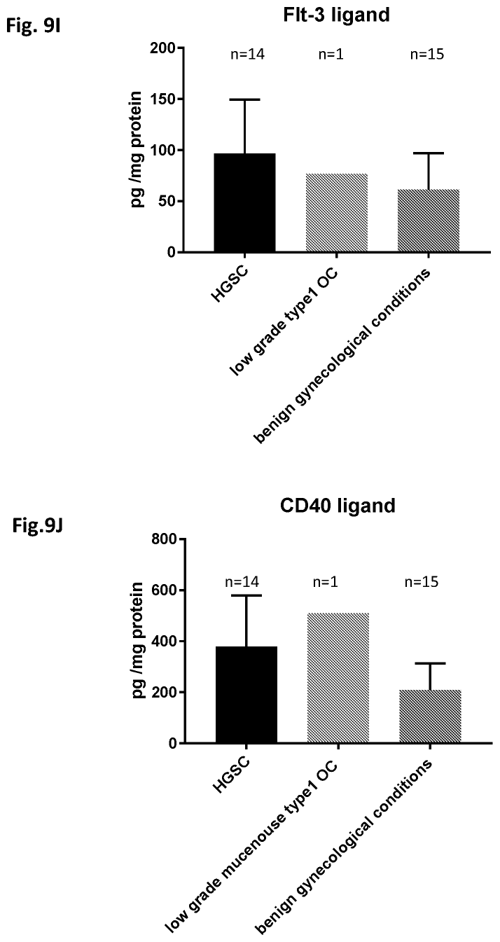

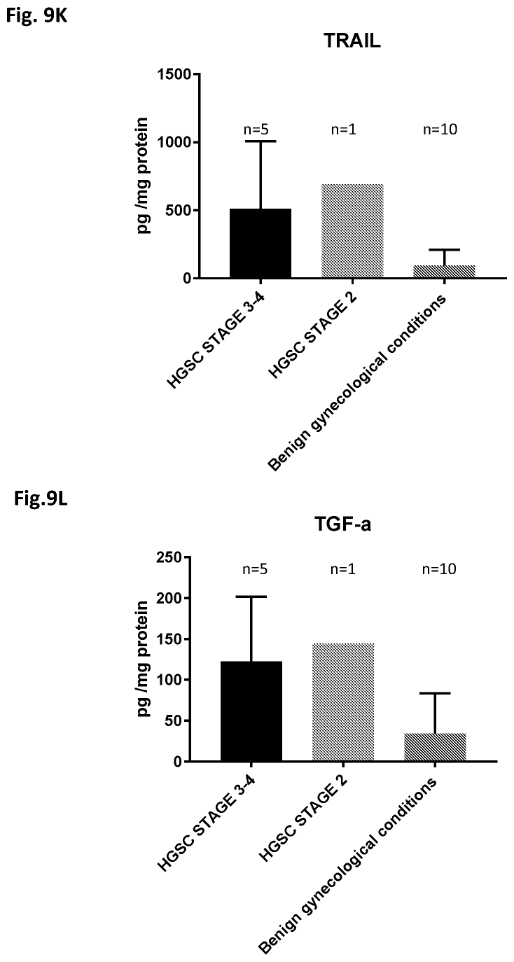

The levels of various biomarkers were determined and compared between the different test groups. The results are presented in FIGS. 9A-L, each presenting the relative expression levels of the respective biomarker in the different test groups.

The results indicate that immune-related biomarkers (such as those presented in FIGS. 9A-L, namely, CD44; IL-8; PD-L1; IL-2; MIP-1b; IL-33; IL-13; Fractalkine; FLT3-Ligand; CD-40 Ligand; TRAIL; and TGF-a, respectively), can differentiate not only between high grade cancer patients and healthy subjects, but also between low grade cancer and healthy subjects.

The results clearly indicate that the immune-related biomarkers can be efficiently used for very early detection of ovarian cancer.

### Example 4—Determining Specific Cancer-Related Biomarker Levels in Vaginal Secretions Samples Obtained from High Stage Epithelial Ovarian Cancer (EOC) Patients, Benign Gynecological Conditions Subjects and Low Grade EOC Patient

Vaginal secretions from healthy subjects (n=18), from high stage epithelial ovarian cancer (EOC) patients (n=18) and from low grade EOC patient were obtained.

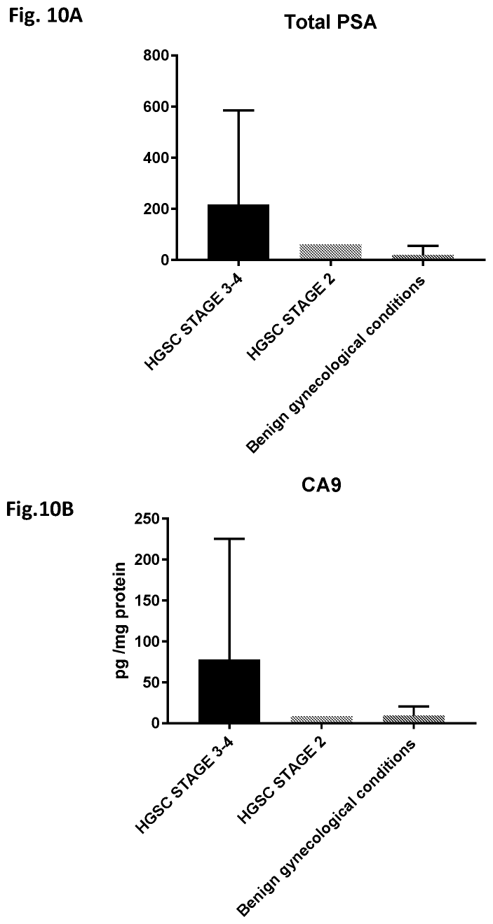

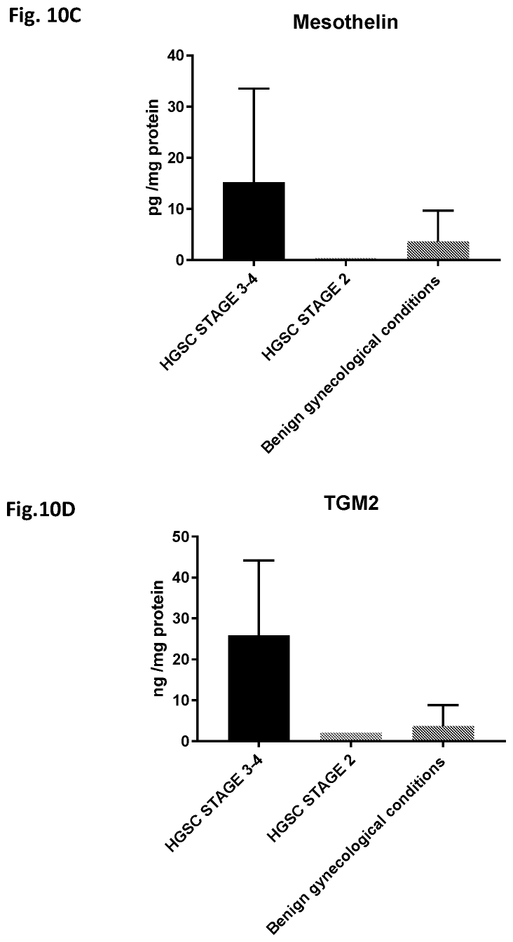

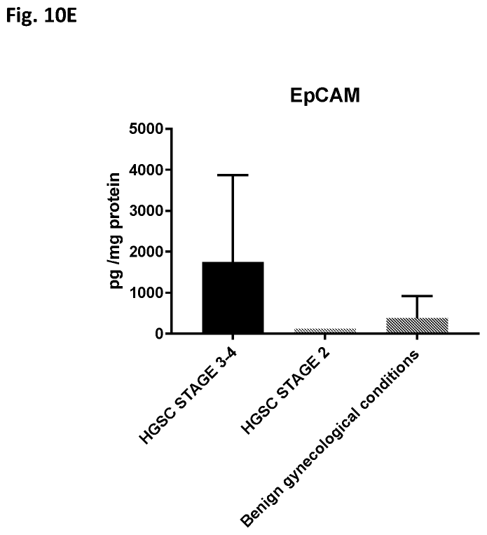

The levels of various biomarkers were determined and compared between the different test groups. The results are presented in FIGS. 10A-E, each presenting the relative expression levels of the respective biomarker in the different test groups.

The results indicate that cancer-related biomarkers (such as those presented in FIGS. 10A-E, namely Total PSA; CA-9; Mesothelin; TGM-2; and EpCAM, respectively), can differentiate between high grade cancer patients and healthy subjects, but do not differentiate between low grade cancer and healthy subjects, as their level does not significantly change in such early stages.

The results clearly indicate that the cancer-related biomarkers can be efficiently used for differentiating between late stage ovarian cancer subjects and healthy subjects, but are not as efficient is differentiating or detecting early stage ovarian cancer.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention. It is to be understood that further trials are being conducted to establish clinical effects.

1.-32. (canceled)

33. A method for treating ovarian cancer in a subject, the method comprising:
obtaining or having obtained a vaginal secretion sample from the subject, and
detecting in the vaginal secretion sample from the subject, at least one biomarker selected from the group consisting of:
Interleukin 2 (IL-2), IL-1a, Interleukin 13 (IL-13), CD-40 Ligand, Flt-3 Ligand, Fractalkine, IL-33, GALECTIN-9, Midkine, MIP-1a, MIP-1b, MIP-3b, PD-L1, RANTES, TGF-alpha, TGM2, Total PSA, TRAIL, Stem Cell factor (SCF), EpCAM, Galectin-1, and Galectin-9; and
treating the subject,
wherein the treating is selected from the group consisting of:
repeating the method; monitoring the subject; administering a therapeutic agent selected from the group consisting of: PARP inhibitor, Olaparib, Rucaparib, Niraparib, Talazoparib, cisplatin, carboplatin, paclitaxel, docetaxel, or any combination thereof;
recommending a medical procedure, recommending no further action; and any combinations thereof;
wherein the detecting comprises contacting the vaginal secretion sample with a reagent specific for the at least one biomarker,
wherein the detection of the at least one biomarker indicates ovarian cancer in the subject.

34. The method according to claim 33, wherein the at least one biomarker is selected from the group consisting of: TRAIL, CD40 ligand, Flt-3 Ligand, Fractalkine, Midkine, TGF-alpha, PDL-1, and TGM2.

35. The method according to claim 34, further comprising detecting at least one additional biomarker selected from the group consisting of: AFP, PSA, CEA, SCF, CD44, Mesothelin, TGM2, EpCAM, Galectin-1, IL-1a, IL-2, IL-8, IL-13, IL-33, IP-10, MIP1a, MIP1b, MIP3b, TNF-a, VEGF; and any combinations thereof.

36. The method according to claim 33, wherein the detecting comprises:
measuring a level of the at least one biomarker in the subject;
comparing the level of the at least one biomarker in the subject to a level of the same at least one biomarker in a control;
determining a change in the level of the at least one biomarker.

37. The method according to claim 36, wherein the method differentiates between advanced stage epithelial ovarian cancer (EOC) and early stage epithelial ovarian cancer.

38. The method according to claim 37, wherein the change in the levels of at least one biomarker selected from the group consisting of: PD-L1, IL-2, MIP-1b, CD-40 Ligand, Flt3-Ligand, TRAIL, TGF-a, IL-13, IL-33, fractalkine, and combinations thereof is indicative of the stage of EOC.

39. The method according to claim 33, comprising detecting at least three biomarkers.

40. The method according to claim 33, wherein the reagent comprises an antibody specific for the at least one biomarker.

41. The method according to claim 33, wherein the treating comprises: administering a therapeutic agent selected from the group consisting of: PARP inhibitor, Olaparib, Rucaparib, Niraparib, Talazoparib, cisplatin, carboplatin, paclitaxel, docetaxel, and any combination thereof.

42. A kit for detecting ovarian cancer in a vaginal secretion sample from a subject, the kit comprising:
one or more reagents specific for a biomarker selected from the group consisting of: Interleukin 2 (IL-2), IL-1a, Interleukin 13 (IL-13), CD-40 Ligand, Flt-3 Ligand, Fractalkine, IL-33, GALECTIN-9, Midkine, MIP-1a, MIP-1b, MIP-3b, PD-L1, RANTES, TGF-alpha, TGM2, Total PSA, TRAIL, Stem Cell factor (SCF), EpCAM, Galectin-1, Galectin-9, and combinations thereof.

43. The kit according to claim 42, wherein the biomarker is selected from the group consisting of: TRAIL, CD40 ligand, Flt-3 Ligand, Fractalkine, Midkine, TGF-alpha, PDL-1, TGM2, and combinations thereof.

44. The kit according to claim 42, wherein the kit further comprises one or more additional reagents specific for a biomarker selected from the group consisting of: AFP, PSA, CEA, SCF, CD44, Mesothelin, TGM2, EpCAM, Galectin-1, IL-1a, IL-2, IL-8, IL-13, IL-33, IP-10, MIP1a, MIP1b, MIP3b, TNF-a, VEGF, and combinations thereof.

45. The kit according to claim 42, wherein the kit comprises one or more reagents specific for at least three biomarkers.

46. The kit according to claim 42, wherein the kit comprises one or more reagents specific for a biomarker selected from the group consisting of: PD-L1, IL-2, MIP-1b, CD-40 Ligand, Flt3-Ligand, TRAIL, TGF-a, IL-13, IL-33, and combinations thereof.

47. The kit according to claim 42, wherein the reagents are antibodies.

48. A kit for early detection of early stage epithelial ovarian cancer (EOC) in a vaginal secretion sample from a subject, the kit comprising:
one or more reagents specific for an immune-related biomarker selected from the group consisting of: PD-L1, IL-2, MIP-1b, CD-40 Ligand, Flt3-Ligand, TRAIL, TGF-a, IL-13, IL-33, fractalkine, and any combinations thereof.

49. The kit according to claim 48, further comprising reagents specific for: CD44 and/or IL-8.

50. The kit according to claim 42, further comprising at least one control for each biomarker,
wherein the control comprises:
a sample from at least one subject not afflicted with cancer or EOC;
a sample comprising a pre-determined amount of the biomarker,
wherein the pre-determined amount of the biomarker is an amount found in at least one subject not afflicted with cancer or EOC.

51. The kit according to claim 48, further comprising at least one control for each biomarker,
wherein the control comprises:
a sample from at least one subject not afflicted with cancer or EOC;
a sample comprising a pre-determined amount of the biomarker,
wherein the pre-determined amount of the biomarker is an amount found in at least one subject not afflicted with cancer or EOC.

52. The kit according to claim 42, further comprising instructions for using the kit to detect ovarian cancer in a vaginal secretion sample from a subject.
